(19) United States
(12) Patent Application Publication
(10) Pub. No.: US 2013/0317098 A1
(43) Pub. Date: Nov. 28, 2013

(54) PROTECTIVE HYDROCOLLOID FOR ACTIVE INGREDIENTS

(71) Applicant: Christian Schaefer, Rheinfelden (DE)

(72) Inventor: Christian Schaefer, Rheinfelden (DE)

(21) Appl. No.: 13/989,601

(22) PCT Filed: Nov. 10, 2011
(86) PCT No.: PCT/EP2011/069862

§ 371 Date: Aug. 7, 2013

(30) Foreign Application Priority Data
Nov. 26, 2010 (CH) 02001/10

(51) Int. Cl. A23J 1/12; A61K 8/64; A23J 3/18; A61K 47/42

(52) U.S. Cl. CPC A23J 1/12; A61K 47/42; A61K 8/645; A23J 3/18
USPC 514/458; 514/773; 514/560; 514/762; 514/691; 514/729; 514/725; 514/703; 530/372; 435/68.1; 426/656; 426/540; 426/72; 426/73; 426/268; 426/442; 426/590; 426/18

(57) ABSTRACT

(Modified) sorghum protein is used as novel protective hydrocolloid for active ingredients, especially fat-soluble active ingredients and/or colorants. Included are compositions comprising (modified) sorghum protein and at least one active ingredient and to their manufacture, as well as to the (modified) sorghum protein itself and its manufacture. These compositions are used for the enrichment, fortification and/or coloration of food, beverages, animal feed, personal care or pharmaceutical compositions, and to food, beverages, animal feed, personal care and pharmaceutical compositions containing such a (modified) sorghum protein and such a composition, respectively.

The present invention is directed to the use of (modified) sorghum protein as novel protective hydrocolloid for active ingredients, especially fat-soluble active ingredients and/or colorants. Moreover, the present invention is directed to compositions comprising (modified) sorghum protein and at least one active ingredient and to their manufacture, as well as to the (modified) sorghum protein itself and its manufacture. The present invention is further directed to the use of such compositions for the enrichment, fortification and/or coloration of food, beverages, animal feed, personal care or pharmaceutical compositions, and to food, beverages, animal feed, personal care and pharmaceutical compositions containing such a (modified) sorghum protein and such a composition, respectively.

Active ingredients, especially fat-soluble active ingredients or colorants, are often not added as such to food, beverages, animal feed, personal care and pharmaceutical compositions, but in form of formulations of the active ingredient in a protective hydrocolloid for reasons of enhancing properties such as chemical stability, (water-)solubility, free-flowing and controlled release etc. Known protective hydrocolloids are e.g. gelatine of different origin (poultry, bovine, pork, fish) and starch. Since protective hydrocolloids of animal origin are often not desired because of religious or allergenic reasons and starch-based protective hydrocolloids might have low preference for consumers who are interested in gluten and corn-free products there is an on-going need for alternative protective hydrocolloids. Nevertheless protective hydrocolloids should have a good nutritional value and a wide range of application fields.

It was now found out that sorghum proteins can be used as such protective hydrocolloids.

Sorghum is an important food crop in many countries. There are several species of sorghum, such as sorghum almum, sorghum amplum, sorghum angustum, sorghum arundinaceum, sorghum bicolour, sorghum bicolor subspecies drummondii (Sudan grass), sorghum brachypodum, sorghum bulbosum, sorghum burmahicum, sorghum ecarinatum, sorghum exstans, sorghum grande, sorghum halepense (Johnson grass), sorghum interjectum, sorghum intrans, sorghum laxiflorum, sorghum leiocladum, sorghum macrospermum, sorghum matarankense, sorghum nitidum, sorghum plumosum, sorghum propinquum, sorghum purpureosericeum, sorghum stipoideum, sorghum timorense, sorghum trichocladum and sorghum versicolor. One of the most common sorghum is sorghum bicolour.

Sorghum bicolor is an important world crop, used for food (as grain and in sorghum syrup or “sorghum molasses”), fodder, the production of alcoholic beverages, as well as biofuels. Most varieties are drought tolerant and heat tolerant, and are especially important in arid regions where the grain is staple or one of the staples for poor and rural people. They form an important component of pastures in many tropical regions. Sorghum is an important food crop in Africa, Central America, and South Asia and is the fifth most important cereal crop grown in the world.

The protein content of sorghum can vary. But it is usually between 9 and 15 weight-%.

Sorghum proteins rank high in nutritional quality. Sorghum proteins are recognized as nutritional and hypoallergenic and can, thus, be a suitable alternative source of protective hydrocolloid for formulations of active ingredients. However, high insolubility and poor functionality of sorghum protein at neutral pH limits its industrial application as a functional ingredient in food and pharmaceuticals products. The present invention overcomes these limitations and incorporates the (modified) sorghum protein as a protective hydrocolloid for formulations of active ingredients, especially of fat-soluble active ingredients and/or colorants.

The predominant sorghum proteins are hydrophobic. The protein can be isolated from the core (endosperm proteins) as well as from the other parts of the sorghum grain.

The extracted proteins are highly insoluble in nature and the conditions used in protein isolation further decrease their solubility, and thus have limited application as a functional ingredient. High-protein sorghum products can be obtained from sorghum flour by alkali extraction followed by precipitation at the isoelectric pH of the protein. Starch-hydrolyzing enzymes such as alpha-amylase, glucoamylase, and pullulanase are often used to separate proteins in sorghum flour by solubilizing and removing starch. In addition to starch hydrolyzing enzymes, cellulase and hemicellulase enzymes have been used to further increase the protein content in sorghum protein concentrate. However, information on suitable extraction methods and functionalities of such isolates is limited. Efficient extraction methods using approved food grade enzymes and chemicals are essential for commercial production and application of sorghum protein.

This need is fulfilled by the compositions of the present invention which comprise a sorghum protein and an active ingredient.

The compositions of the present invention may be solid compositions, i.e. stable, water-soluble or water-dispersible powders, or they may be liquid compositions, i.e. aqueous colloidal solutions or oil-in-water dispersions of the aforementioned powders. The stabilised oil-in-water dispersions, which may be oil-in-water emulsions or may feature a mixture of suspended, i.e. solid, particles and emulsified, i.e. liquid, droplets, may be prepared by the methods described below or by an analogous manner.

More specifically, the present invention is concerned with stable compositions in powder form comprising one or more (fat-soluble) active ingredients and/or one or more colorants in a matrix of a (modified) sorghum protein.

Preferably the amount of the (modified) sorghum protein is from 1 to 70 weight-%, more preferably from 5 to 50 weight-%, even more preferably from 10 to 40 weight-%, most preferably from 10 to 20 weight-% (with 20 weight-% being the most preferred one) and/or the amount of the (fat-soluble) active ingredient and/or colorant is from 0.1 to 90 weight-%, preferably from 1 to 80 weight-%, more preferably from 1 to 20 weight-%, based on the total amount of the composition. If additional adjuvants and/or excipients such as tocopherol and/or ascorbyl palmitate are present, they are present in an amount of from 0.01 to 50 weight-%, preferably in an amount of from 0.1 to 30 weight-%, more preferably in an amount of from 0.5 to 10 weight-%, based on the total amount of the composition.

### (MODIFIED) SORGHUM PROTEIN

In preferred embodiments of the present invention the sorghum protein is a modified sorghum protein whose manufacture is described below. An especially preferred sorghum protein is one obtained by the following steps: alkaline extraction, (enzymatically modification, especially with Alkalase), centrifugation and ultra-filtration. If needed for the further use the thus obtained modified sorghum protein may also be dried.

The invention is, however, not restricted to the use of such manufactured modified sorghum proteins.

Even more preferred are (modified) sorghum proteins that have an emulsion capacity of ≧220 ml/g, preferably of ≧350 ml/g, more preferably of ≧500 ml/g, even more preferably of from 500 ml/g to 1000 ml/g. Additionally in preferred embodiments of the invention the used (modified) sorghum proteins have an emulsion activity of ≧0.2, preferably of ≧0.45, more preferably of ≧0.5, even more preferably of from 0.5 to 1.0. The determination of the emulsion capacity as well as the determination of the emulsion activity are described below. The present invention refers also to these (modified) sorghum proteins themselves.

### Determination of the Emulsion Capacity

The emulsion capacity of the (modified) sorghum protein can be determined according to the method of Gbogouri et al, Journal of Food Science 2004, Vol. 69, Nr. 8, 615, based on oil titration. Dispersions (0.1% w/w, 50 mL, pH 7.0) of the (modified) sorghum protein are prepared in deionized water. The protein solution is homogenized with a homogenizer at setting 1 (Virtishear Tempest, The Virtis Co., Gardiner, N.Y., U.S.A.). Corn oil is added into the protein solution with a flow rate of about 17 g/min using a peristaltic pump. The conductivity of the emulsion is recorded continuously by a conductivity-meter and used as a parameter for the determination of the inversion point of the emulsion. The amount of oil added to the inversion point is used to calculate the emulsifying capacity. The emulsifying capacity is expressed as the ratio of emulsified oil minus the blank over the amount of proteins in sample. The blank is the quantity of oil added before the phase inversion in 50 mL of deionized water.

Alternatively, the emulsion capacity can be determined according to the method of Vuillemard and others based on oil titration. Protein dispersions (0.5% w/w, 40 mL, pH 7.0) are prepared in distilled water. The protein solution is homogenized with homogenizer at setting 6 (Virtishear Tempest, The Virtis Co., Gardiner, N.Y., U.S.A.). Corn oil is added into the protein solution at about 12 g/min flow rate using a pump. The conductivity of the emulsion is recorded continuously by a conductivity-meter and used as a parameter for the determination of the inversion point of the emulsion. The amount of oil that added up to the inversion point is used to calculate the emulsifying capacity. Emulsifying capacity is expressed as the ratio of emulsified oil minus the blank over the amount of proteins in sample. The blank is the quantity of oil added before the phase inversion in 40 mL distilled water.

### Determination of the Emulsion Activity

The emulsion activity is determined by the turbidimetric method of Pearce and Kinsella, Journal of Agric Food Chem. 1978, 26:716-722. A mixture of 6 ml of a 0.1% solution of the (modified) sorghum protein in 10 mM phosphate buffer of a pH of 7.0 and 2 ml of corn oil is homogenized for 1 minute with a sonicator at setting 6 (Virtishear Tempest, The Virtis Co., Gardiner, N.Y., U.S.A.). 50 microliters of the mixture are transferred into 5 ml of an 0.1% aqueous solution of SDS (w/v) 0 and 10 minutes after the homogenization. The absorbance of the solution at 500 nm is determined with a spectrometer (Shimadzu Model UV-1601, Kyoto, Japan). The absorbance at the time 0 after homogenization is the emulsion activity of the (modified) sorghum protein.

In further preferred compositions of the present invention the (modified) sorghum protein is cross-linked with at least one compound selected from the group consisting of reducing sugars, glycoproteins and glycopeptides.

### Active Ingredient

The active ingredients are those ingredients with a pharmacological effect or those providing health benefits to the human or animal body in general. Preferably the active ingredient is a fat-soluble active ingredient and/or a colorant.

The fat-soluble active ingredient and/or the colorant is preferably selected from the group consisting of carotenes and structurally related polyene compounds, fat-soluble vitamins, coenzyme Q10, polyunsaturated fatty acids such as eicosapentaenoic acid (EPA) and docosahexaenoic acid (DHA) and esters thereof (such as the ethyl esters or the triglycerides (containing the same or different fatty acids)), mono-, di-, triglycerides rich in polyunsaturated fatty acids, fat-soluble UV-A filters, UV-B filters, as well as their physiologically acceptable derivatives such as their esters, especially with C₁₋₂₀ carbonic acids, and any mixtures of them.

The most preferred fat-soluble vitamins are vitamin A or vitamin E (as well as their derivatives).

Preferred examples of the carotenes and structurally related polyene compounds are carotenoids such as α-carotene, β-carotene, 8′-apo-β-carotenal, 8′-apo-βcarotenoic acid esters such as the ethyl ester, canthaxanthin, astaxanthin, lycopene, lutein, zeaxanthin, crocetin, α-zeacarotene, β-zeacarotene, as well as their physiologically acceptable derivatives such as their esters, especially with C₁₋₂₀ carbonic acids, and any mixtures of them.

The most preferred carotenoid is β-carotene.

The term “β-carotene” encompasses the all-cis as well as the all-trans isomers and all possible mixed cis-trans-isomers. The same applies for the other carotenoids.

The term “zeaxanthin” encompasses the natural R,R-zeaxanthin, as well as S,S-zeaxanthin, meso-zeaxanthin and any mixture of them. The same applies for lutein.

The (fat-soluble) active ingredients may be of natural origin, i.e. isolated/extracted from plants, purified and/or concentrated, as well as those synthesized by chemical and/or microbiological (fermentative) routes.

### Further Components

Beside the active ingredient and the (modified) sorghum protein the compositions of the present invention may preferably additionally contain at least one water-soluble antioxidant and/or fat-soluble antioxidant.

The water-soluble antioxidant may be for example ascorbic acid or a salt thereof, preferably sodium ascorbate, watersoluble polyphenols such as hydroxytyrosol and oleuropein aglycon; epigallocatechingallate (EGCG) or extracts of rosemary or olives.

The fat-soluble antioxidant may be for example a tocopherol, e.g. dl-α-tocopherol (i.e. synthetic tocopherol), d-α-tocopherol (i.e. natural tocopherol), β- or γ-tocopherol, or a mixture of two or more of these; butylated hydroxytoluene (BHT); butylated hydroxyanisole (BHA); ethoxyquin, propyl gallate; tert. butyl hydroxyquinoline; or 6-ethoxy-1,2-di-hydroxy-2,2,4-trimethylquinoline (EMQ), or an ascorbic acid ester of a fatty acid, preferably ascorbyl palmitate or stearate.

The compositions of the present invention may further contain a co-emulgator selected from the group consisting of mono- and diglycerides of fatty acids, polyglycerol esters of fatty acids, lecithins; N-acylated amino acids and derivatives thereof, N-acylated peptides with an alkyl or alkenyl radical, and salts thereof; alkyl or alkenyl ether or ester sulfates, and derivatives and salts thereof; polyoxyethylenated alkyl or alkenyl fatty ethers or esters; polyoxyethylenated alkyl or alkenyl carboxylic acids and salts thereof; N-alkyl or N-alkenyl betaines; alkyl-trimethylammonium or alkenyltrimethylammonium and salts thereof; polyol alkyl or alkenyl ether or ester; and mixtures thereof.

Preferred examples of polyol alkyl or alkenyl ethers or esters are sorbitan alkyl or alkenyl esters polyoxyethylenated with at least 20 units of ethylene oxide, such as sorbitan palmitate 20 EO or Polysorbate 40 marketed under the tradename Montanox 40 DF by the company Seppic, sorbitan laurate 20 EO or Polysorbate 20 marketed under the tradename Tween 20 by the company ICI, and sorbitan monostearate.

The formulations according to the present invention may further be pressed into tablets, whereby one or more excipients and/or adjuvants selected from the group consisting of monosaccharides, disaccharides, oligosaccharides and polysaccharides, glycerol, and triglycerides, may be added.

Preferred examples of mono- and disaccharides which may be present in the compositions of the present invention are sucrose, invert sugar, xylose, glucose, fructose, lactose, maltose, saccharose and sugar alcohols.

Preferred examples of the oligo- and polysaccharides are starch, modified starch and starch hydrolysates. Preferred examples of starch hydrolysates are dextrins and maltodextrins, especially those having the range of 5 to 65 dextrose equivalents (DE), and glucose syrup, especially such having the range of 20 to 95 DE. The term “dextrose equivalent” (DE) denotes the degree of hydrolysis and is a measure of the amount of reducing sugar calculated as D-glucose based on dry weight; the scale is based on native starch having a DE close to 0 and glucose having a DE of 100.

The triglyceride is suitably a vegetable oil or fat, preferably corn oil, sunflower oil, soybean oil, safflower oil, rapeseed oil, peanut oil, palm oil, palm kernel oil, cotton seed oil, olive oil or coconut oil.

Solid compositions may in addition contain an anti-caking agent, such as silicic acid or tricalcium phosphate and the like, and up to 10 weight-%, as a rule 2 to 5 weight-%, of water.

### Manufacture of the Composition

An object of the present invention is also a process for the manufacture of the composition of the present invention which comprises the following steps:
I) preparing an aqueous solution or colloidal solution of a (modified) sorghum protein,
II) optionally adding at least a water-soluble excipient and/or adjuvant to the solution prepared in step I),
III) preparing a solution or dispersion of at least an active ingredient, preferably of at least a fat-soluble active ingredient and/or colorant, and optionally at least a fat-soluble adjuvant and/or excipient,
IV) mixing the solutions prepared in step I) to III) with each other,
V) homogenising the thus resulting mixture,
VI) optionally adding a cross-linking agent for cross-linking the (modified) sorghum protein,
VIa) optionally submitting the mixture resulting after having performed step VI) to enzymatic treatment or heat treatment to cross-link the (modified) sorghum protein
VII) optionally converting the dispersion obtained in step V) and/or VI) into a powder,
VIII) optionally drying the powder obtained in step VII),
IX) optionally submitting the dry powder to heat treatment or to enzymatic treatment to cross-link the (modified) sorghum protein,

with the proviso that only step VIa) or step IX) is carried out, but not both, when step VI) is carried out.

As an alternative method it is also possible to crosslink the protein after the modification but before the emulsion step.

### Step I

This step is simply performed by adding water to the (modified) sorghum protein or vice versa, optionally under stirring. Alternatively homogenization may be possible via ultrasonication.

Preferably the (modified) sorghum protein with the preferences as described above is used.

### Step II

Water-soluble excipients and/or adjuvants that may be added are e.g. monosaccharides, disaccharides, oligosaccharides and polysaccharides, glycerol and water-soluble antioxidants. Examples of them are given above.

### Step III

Active ingredients are those as described above.

The (fat-soluble) active ingredient and/or colorant and optional fat-soluble excipients and adjuvents are either used as such or dissolved or suspended in a triglyceride and/or an (organic) solvent.

Suitable organic solvents are halogenated aliphatic hydrocarbons, aliphatic ethers, aliphatic and cyclic carbonates, aliphatic esters and cyclic esters (lactones), aliphatic and cyclic ketones, aliphatic alcohols and mixtures thereof.

Examples of halogenated aliphatic hydrocarbons are mono- or polyhalogenated linear, branched or cyclic C₁- to C₁₅-alkanes. Especially preferred examples are mono- or polychlorinated or -brominated linear, branched or cyclic C₁- to C₁₅-alkanes. More preferred are mono- or polychlorinated linear, branched or cyclic C₁- to C₁₅-alkanes. Most preferred are methylene chloride and chloroform.

Examples of aliphatic esters and cyclic esters (lactones) are ethyl acetate, isopropyl acetate and n-butyl acetate; and γ-butyrolactone.

Examples of aliphatic and cyclic ketones are acetone, diethyl ketone and isobutyl methyl ketone; and cyclopentanone and isophorone.

Examples of cyclic carbonates are especially ethylene carbonate and propylene carbonate and mixtures thereof.

Examples of aliphatic ethers are dialkyl ethers, where the alkyl moiety has 1 to 4 carbon atoms. One preferred example is dimethyl ether.

Examples of aliphatic alcohols are ethanol, iso-propanol, propanol and butanol.

Furthermore any oil (triglycerides), orange oil, limonen or the like and water can be used as a solvent.

Fat-soluble excipients and/or adjuvants that may be added are e.g. corn oil, mono- or diglycerides of fatty acids, polyglycerol fatty acids, and middle chain triglycerides (“MCT”).

### Step IV

In an alternative process of the present invention step III) is not carried out, but the active ingredient and the optional fat-soluble excipient and/or adjuvant is directly added to the solution of step I) or II).

### Step V

For the homogenisation conventional technologies, such as high-pressure homogenisation, high shear emulsification (rotor-stator systems), micronisation, wet milling, microchanel emulsification, membrane emulsification or ultrasonification can be applied. Other techniques used for the preparation of compositions containing (fat-soluble) active ingredients and/or colorant for enrichment fortification and/or coloration of food, beverages, animal feed, cosmetics or pharmaceutical compositions are disclosed in EP-A 0 937 412 (especially paragraphs [0008], [0014], [0015], [0022] to [0028]), EP-A 1 008 380 (especially paragraphs [0005], [0007], [0008], [0012], [0022], [0023] to [0039]) and in U.S. Pat. No. 6,093,348 (especially column 2, line 24 to column 3, line 32; column 3, line 48 to 65; column 4, line 53 to column 6, line 60), the contents of which are incorporated herein by reference.

### Step VI

The cross-linking agent is preferably selected from the group consisting of reducing sugars, glycoproteins, and glycopeptides. Thus an intermolecular cross-linking between the (modified) sorghum protein and the sugar or sugar part of the glycoprotein/glycopeptide is formed. Preferred examples of the cross-linking agent are the reducing sugars such as glucose, fructose, saccharose and xylose.

### Step VIa

The cross-linking can be achieved by submitting mixtures additionally containing a cross-linking agent as described above to heat-treatment to cause cross-linking of the sugar with the protein in a Maillard type reaction, i.e. by thermally treatment, preferably at temperatures from about 30° C. to about 160° C., more preferably at temperatures form about 70° C. to about 100° C., most preferably at temperatures from about 80° C. to about 90° C.

Cross-linking of the (modified) sorghum protein with the cross-linking agent can also be achieved by treatment with cross-linking enzymes (acyltransferases, EC 2.3, e.g. transglutaminase, EC 2.3.2.13, protein-glutamine:y-glutamyltransferase), i.e. by enzymatically treatment, conveniently carried out at temperatures from about 0 to about 70° C., preferably at temperatures from about 20° C. to about 40° C. Preferably the enzymatic treatment according to step Via) is a treatment with a cross-linking enzyme, particularly with a transglutaminase.

Enzymatic cross-linking results in stable protein-containing polysaccharide networks, in the case of a transglutaminase by the formation of ε-(γ-glutamyl)-lysine isopeptide bonds. The use of glycoproteins or glycopeptides is preferred for the enzymatic cross-linking.

Both techniques, heat-treatment to cause cross-linking of the sugar with the protein in a Maillard type reaction and enzymatic cross-linking can be used for the incorporation of lipophilic moieties and can be carried out either in a dried form of the composition (step IX), or in an aqueous solution or suspension (step VIa). The enzymatic cross-linking is preferably carried out in an aqueous solution or suspension.

### Step VII

The so-obtained dispersion, which is an oil-in-water dispersion, can be converted after removal of the organic solvent (if present) into a solid composition, e.g. a dry powder, using any conventional technology such as spray drying, spray drying in combination with fluidised bed granulation (the latter technique commonly known as fluidised spray drying or FSD), or by a powder-catch technique whereby sprayed emulsion droplets are caught in a bed of an absorbent, such as starch, calcium silicate and silicon dioxide, and subsequently dried.

Spray-drying may be performed at an inlet-temperature of from about 100° C. to about 250° C., preferably of from about 150° C. to about 200° C., more preferably of from about 160° C. to about 190° C., and/or at an outlet-temperature (product temperature) of from about 45° C. to about 160° C., preferably of from about 55° C. to about 110° C., more preferably of from about 65° C. to about 95° C.

### Step VIII

The drying of the powder obtained in step VII is preferably carried out at a temperature of ≦100° C., preferably at a temperature of from 20 to 100° C., more preferably at a temperature of from 60 to 70° C. If the drying is performed in vacuum the temperature is lower.

### Step IX

The cross-linking via heat-treatment is carried out as already described above for step VIa. The same applies for the enzymatic treatment, which is, however, preferably carried out in solution/suspension.

### Manufacture of the (Modified) Sorghum Protein

The present invention is also directed to a process for the manufacture of a (modified) sorghum protein starting from milled sorghum, comprising the following steps a) to e) with the proviso that at least one of the steps b), c) and d) is carried out. The sorghum bran can be removed before milling in case a pure endosperm protein should be obtained.

The process comprises:
a) preparing an aqueous solution or suspension of milled sorghum (whereby the sorghum bran can be removed before milling) whereby the solution or suspension preferably has a dry mass content of from 0.1 to 30 weight-%, preferably from 10 to 15 weight-%, based on the total amount of the aqueous solution or suspension;
b) optionally removing the non-protein part or the protein part of the milled sorghum (whereby the sorghum bran can be removed before milling) to obtain the sorghum protein;
c) optionally modifying the protein part of the milled sorghum(whereby the sorghum bran can be removed before milling) to obtain modified sorghum protein;
d) optionally isolating the (modified) sorghum protein;
e) optionally converting the (modified) sorghum protein into a solid form.

In the context of the present invention “sorghum protein” means especially the product obtained by performing either steps a) and b); or steps a) and d); or steps a), b) and e); or steps a), b) and d); or steps a), d) and e); or steps a), b), d) and e). Preferred are the embodiments where steps a) and b) (and d) and/or e)) are performed, especially preferred are the embodiments where steps a), b) and d) (and e)) are performed.

In the context of the present invention “modified sorghum protein” means especially the product where step c) is carried out, i.e. the product obtained by performing either steps a) and c); or steps a), b) and c); or steps a), c) and d); or steps a), c) and e); or steps a), b) c) and d); or steps a), c), d) and e); or steps a), b), c) and e); or steps a), b), c), d) and e).

### Step a)

Milled sorghum, where the sorghum bran was removed before milling, is also known under the expression “sorghum flour”.

This step is simply performed by adding water to the sorghum flour or vice versa, optionally by stirring vigorously (with a mechanical stirrer) until the sorghum flour is completely dispersed, or by homogenizing the sorghum flour suspension with a homogenizer, e.g. for 5 minutes at room temperature.

### Step b)

Removing of the non-protein part

Step b) may preferably be achieved by treating the sorghum flour with non-protein degrading enzymes, e.g. with a 0.5% aqueous suspension of Termamyl® at a temperature of 90° C. for 2 hours and then with a 0.1% aqueous suspension of a cellulase at a temperature of 50° C. for 30 minutes—without any pH adjustment (pH 6-7), deactivating the enzymes, separating and removing the non-protein part from the protein part of the sorghum flour.

Preferred examples of non-protein degrading enzymes are starch-degrading enzymes such as α-amylases and cellulases, i.e. cellulose-degrading enzymes, and mixtures thereof. A preferred example of an α-amylase is Termamyl® 120, Type L, commercially available from Novo Nordisk Biochem, North America, Inc., USA. Other preferred examples are Liquzyme® Supra, commercially available from Novo Nordisk Biochem, North America, Inc., USA, Amylase S “Amano” 35 G, commercially available from Amano Pharmaceutical Co. Ltd., Nagoya, Japan, Multifect Cellulase, commercially available from Genencor International, Inc., USA, and Cellulase T “Amano” 4, commercially available from Amano Pharmaceutical Co. Ltd., Nagoya, Japan.

The reaction of the enzymes can be stopped by neutralising the solution or suspension if an inorganic acid (e.g. hydrochloric acid) or an organic acid (e.g. citric acid) or base is used or by heating to denature the enzymes.

The denaturation may be achieved by heating the solution to a temperature of from 80 to 95° C., preferably to a temperature of from 80 to 85° C. (especially at a low pH of from 3.5 to 4.5) for 10 to 15 minutes. Afterwards the solution may be cooled to 50° C.

The separation of the non-protein part may be achieved by centrifugation (5000 g for 15 minutes) (whereby the non-protein part is in the water phase), followed by washing with deionized water. The sorghum protein remains in pellets.

Removing of the protein part

Alternatively a so-called “alkaline extraction” or a so-called “salt-extraction” may be performed before the centrifugation or filtration.

“Alkaline extraction” means that first the pH of the solution or suspension of the sorghum flour is adjusted to a value of from 7 to 12, preferably to a value of from 8 to 10, more preferably to a value of about 9, with an alkali solution (e.g. an aqueous NaOH solution) at 40 to 60° C. for 3 hours.

In cases where the protein yield is more important than the protein functionality it may be advantageous to adjust the pH preferably to a value of from 8 to 12, more preferably of from 9 to 12, even more preferably from 10 to 12.

Preferably such a base has a concentration of about 0.1 to 5 M, preferably of about 0.5 to about 2 M. The base may be an inorganic base. Examples of inorganic bases are (earth) alkali hydroxides such as sodium hydroxide (preferred), potassium hydroxide and calcium hydroxide.

A “salt-extraction” is similar to an “alkali-extraction”, but in addition to the base a salt such as sodium chloride is used. In a preferred embodiment of the invention an aqueous 0.08 M sodium chloride solution (adjusted to pH 11 with NaOH) is used as the extracting solvent.

In both cases (alkaline or salt extraction) the protein part is transferred to the water phase. The protein part may be separated then by centrifugation or filtration from the non-protein part.

### Step c)

The modification of the sorghum flour may be achieved by treating it(s protein part) with (commercially available) food grade alkaline, neutral and/or acid proteases. For some proteases the enzyme specifications and the optimum conditions are given in the examples.

The proteases may be from bacteria or fungi, as well as from fruit or may have animal origin.

Examples of alkaline proteases are the commercially available Alkalase® (Novo Nordisk Biochem, Franklinton, N.C., USA), Alkaline protease® (Enzyme Development Corporation, New York, N.Y., USA), Protex 6L® (Genencor® Bacterial Alkaline Protease, Genencor International, Inc., Rochester, N.Y., USA) and Genencor® Protease 899 (Genencor International, Inc., Rochester, N.Y., USA).

Examples of neutral proteases are the commercially available Bromelain® (Enzyme Development Corporation, New York, N.Y., USA), Liquipanol® (Enzyme Development Corporation, New York, N.Y., USA) and bacterial neutral-protease (Genencor International, Inc., Rochester, N.Y., USA). A further example of a neutral protease is the commercially available Collupilin® of DSM Food Beverages, Delft, Netherlands, produced from Carica papaya, a plant, i.e. an enzyme of fruit origin.

Examples of acid proteases are pepsin (Sigma, USA) and Acid protease (Amano Pharmaceutical Co. Ltd., Nagoya, Japan).

In a preferred embodiment of the process of the present invention the protein part of the sorghum flour is treated subsequently by two different alkaline proteases at a pH range of from 7 to 10 for 10 to 80 minutes at 40 to 60° C.

Preferably one of these proteases is a serine specific protease such as Alkalase®, Protex 6L® or Alkaline protease® and the other is a cysteine specific protease such as Liquipanol® or Bromelain®.

The step may also be modified by not adding the enzyme(s) at once but by adding them (subsequently or simultaneously) portion wise.

Step c) may also be performed after step d), i.e. first the sorghum protein is isolated and then it is modified.

### Step d)

Step d) is preferably carried out by centrifugation and/or filtration, preferably by ultrafiltration. The ultrafiltration may be carried out without prior centrifugation.

After enzyme inactivation, the hydrolyate may be preferably centrifuged at low speed (1000 g for 10 minutes) to separate insoluble proteins and impurities.

The soluble fractions of the protein hydrolysates may then be filtered through Whatman #0.4 filter paper and the filtrate be subjected to a sequential ultrafiltration (UF) and diafiltration (DF) with membranes with a molecular weight cut-off (MWCO) of ≧5 kDa, preferably with membranes with a molecular weight cut-off (MWCO) of ≧30 kDa, more preferably with membranes with a molecular weight cut-off (MWCO) of ≧50 kDa, most preferably with membranes with a molecular weight cut-off (MWCO) of from 50 to 750 kDa.

Ultrafiltration and diafiltration may be performed at room temperature with an inlet pressure of from 20 to 25 psi and an outlet-pressure of 10 psi. The solution may then be ultrafiltrated to a concentration factor of 5. Immediately after ultrafiltration, the retenate may be diafiltrated two times with twice the volume of deionized water. During ultrafiltration and diafiltration the pH of the solution may be maintained between pH 8.0 and 9.0 in order to keep the protein soluble.

### Step e)

The conversion into a solid form, e.g. a dry powder, can be achieved by any drying method known to the person skilled in the art. Preferred are spray drying or freeze-drying. Spray drying is preferably performed at an inlet temperature of about 200° C. to about 210° C. and at an outlet temperature of about 70° C. to about 75° C. The freeze-drying is preferably performed at a temperature of from about −20° C. to about −50° C. for 10 to 48 hours.

An object of the present invention is also the (modified) sorghum protein obtainable by any process as described above.

### INDUSTRIAL APPLICABILITY

The present invention is directed to the use of a composition as described above for the enrichment, fortification and/or coloration of food, beverages, animal feed, personal care or pharmaceutical compositions, as well as to the food, beverages, animal feed, personal care and pharmaceutical compositions containing such a composition as described above themselves.

The present invention is also directed to food, beverages, animal feed, personal care and pharmaceutical compositions containing a (modified) sorghum protein as described above, as well as to the use of such a (modified) sorghum protein, preferably such as described above, as protective hydrocolloid for active ingredients, especially fat-soluble active ingredients and/or colorants.

Animals including humans in the context of the present invention encompass besides humans especially farm animals such as sheep, cow, horses, poultry (broiler and egg pigmentation), shrimps and fish (especially salmon and rainbow trout) as well as pets such as cat, dogs, birds (e.g. flamingos) and fish.

Beverages wherein the compositions of the present invention can be used, especially as a colorant or a functional ingredient, can be carbonated beverages e.g., flavoured seltzer waters, soft drinks or mineral drinks, as well as non-carbonated beverages e.g. flavoured waters, fruit juices, fruit punches and concentrated forms of these beverages. They may be based on natural fruit or vegetable juices or on artificial flavours. Also included are alcoholic beverages and instant beverage powders. Besides, sugar containing beverages, diet beverages with non-caloric and artificial sweeteners are also included.

Further, dairy products, obtained from natural sources or synthetic, are within the scope of the food products wherein the compositions of the present invention can be used, especially as a colorant or as a functional ingredient. Typical examples of such products are milk drinks, ice cream, cheese, yoghurt and the like. Milk replacing products such as soymilk drinks and tofu products are also comprised within this range of application.

Also included are sweets which contain the compositions of the present invention as a colorant or as a functional ingredient, such as confectionery products, candies, gums, desserts, e.g. ice cream, jellies, puddings, instant pudding powders and the like.

Also included are cereals, snacks, cookies, pasta, soups and sauces, mayonnaise, salad dressings and the like which contain the compositions of the present invention as a colorant or a functional ingredient. Furthermore, fruit preparations used for dairy and cereals are also included.

The final concentration of the (fat-soluble) active ingredient and/or the colorant which is added via the compositions of the present invention to the food products may be from 0.1 to 500 ppm, particularly from 1 to 50 ppm, based on the total weight of the food composition and depending on the particular food product to be coloured or fortified and the intended grade of coloration or fortification.

The food compositions of this invention are preferably obtained by adding to a food product the (fat-soluble) active ingredient and/or the colorant in the form of a composition of this invention. For coloration or fortification of a food or a pharmaceutical product a composition of this invention can be used according to methods per se known for the application of water dispersible solid compositions of the present invention.

In general the composition may be added either as an aqueous stock solution, a dry powder mix or a pre-blend with other suitable food ingredients according to the specific application. Mixing can be done e.g. using a dry powder blender, a low shear mixer, a high-pressure homogeniser or a high shear mixer depending on the formulation of the final application. As will be readily apparent such technicalities are within the skill of the expert.

Pharmaceutical compositions such as tablets or capsules wherein the compositions are used as a colorant are also within the scope of the present invention. The coloration of tablets can be accomplished by adding the compositions of the present invention in form of a liquid or solid colorant composition separately to the tablet coating mixture or by adding a colorant composition to one of the components of the tablet coating mixture. Coloured hard or soft-shell capsules can be prepared by incorporating a colorant composition in the aqueous solution of the capsule mass.

Pharmaceutical compositions such as tablets such as chewable tablets, effervescent tablets or filmcoated tablets or capsules such as hard shell capsules wherein the compositions are used as an active ingredient are also within the scope of the present invention. The compositions of the present invention are typically added as powders to the tableting mixture or filled into the capsules in a manner per se known for the production of capsules.

Animal feed products such as premixes of nutritional ingredients, compound feeds, milk replacers, liquid diets or feed preparations wherein the compositions are either used as a colorant for pigmentation e.g. for egg yolks, table poultry, broilers or aquatic animals (especially shrimps, salmon, rainbow trout) or as an active ingredient are also within the scope of the present invention.

Personal care compositions: Cosmetics, toiletries and derma products i.e. skin and hair care products such as creams, lotions, baths, lipsticks, shampoos, conditioners, sprays or gels wherein the compositions are used as a colorant or as an active ingredient are also within the scope of the present invention.

1. A composition comprising a sorghum protein and an active ingredient.

2. The composition according to claim 1, wherein the sorghum protein is a modified sorghum protein.

3. The composition according to claim 1, wherein the active ingredient is a fat-soluble active ingredient and/or a colorant.

4. The composition according to claim 1, Wherein the fat-soluble active ingredient and/or the colorant is a carotene or a structurally related polyene compound, a fat soluble vitamin, a triglyceride rich in polyunsaturated fatty acids, an oil soluble UV-A filter, an UV-B filter or a mixture thereof.

5. The composition according to claim 4, wherein the carotene or structurally related polyene compound is a carotenoid such as α-carotene, β-carotene, 8′-apo-β-carotenal, 8′-apo-β-carotenoic acid esters, canthaxanthin, astaxanthin, lycopene, lutein, zeaxanthin, crocetin, α-zeacarotene, β-zeacarotene or a mixture thereof.

6. The composition according to claim 5, wherein the carotenoid is β-carotene.

7. The composition as in claim 4, wherein the fat-soluble vitamin is Vitamin A or E.

8. A process for the manufacture of a composition as claimed in claim 1 which comprises the following steps:
I) preparing an aqueous solution or colloidal solution of a (modified) sorghum protein,
II) optionally adding at least a water-soluble excipient and/or adjuvant to the solution prepared in step I),
III) preparing a solution or dispersion of at least an active ingredient, preferably of at least a fat-soluble active ingredient and/or colorant, and optionally at least a fat-soluble adjuvant and/or excipient,
IV) mixing the solutions prepared in step I) to III) with each other,
V) homogenising the thus resulting mixture,
VI) optionally adding a cross-linking agent for cross-linking the (modified) sorghum protein,
VIa) optionally submitting the mixture resulting after having performed step VI) to enzymatic treatment or heat treatment to cross-link the (modified) sorghum protein
VII) optionally converting the dispersion obtained in step V) and/or VI) into a powder,
VIII) optionally drying the powder obtained in step VII),
IX) optionally submitting the (dry) powder to heat treatment or to enzymatic treatment to cross-link the (modified) sorghum protein,
with the proviso that only step VIa) or step IX) is carried out, but not both, when step VI) is carried out.

9. A (modified) sorghum protein obtainable by a process according to claim 8.

10. Use of a composition as claimed in claim 1 for the enrichment, fortification and/or coloration of food, beverages, animal feed, personal care or pharmaceutical compositions.

11. Food, beverages, animal feed, personal care and pharmaceutical compositions containing a composition as claimed in claim 1.
